(19) United States
(12) Patent Application Publication
(10) Pub. No.: US 2014/0255524 A1
(43) Pub. Date: Sep. 11, 2014

(54) ALPHA-GPC DIETARY SUPPLEMENTS FOR WEIGHT LOSS AND/OR INCREASING OR MAINTAINING LEAN BODY MASS

(71) Applicant: Pipeline Nutrition, Inc., Lutz, FL (US)

(72) Inventor: KC QUINTANA, Tampa, FL (US)

(21) Appl. No.: 14/201,690

(22) Filed: Mar. 7, 2014

Related U.S. Application Data
(60) Provisional application No. 61/775,456, filed on Mar. 8, 2013.

(51) Int. Cl. A61K 31/661; A61K 45/06

(52) U.S. Cl. CPC A61K 31/661; A61K 45/06
USPC 424/727; 514/114

(57) ABSTRACT

Disclosed herein are methods for weight loss and/or maintaining weight and/or increasing and/or maintaining lean body mass using glycerylphosphorylcholine (alpha-GPC). Disclosed herein are formulations of glycerylphosphorylcholine (alpha-GPC) useful for weight loss, and/or maintaining weight and/or maintaining and/or increasing lean muscle mass.

### RELATED APPLICATIONS

This application claims priority to and the benefit of U.S. Ser. No. 61/775,456; filed Mar. 8, 2013, which is incorporated herein by reference in its entirety and for all purposes.

### FIELD OF THE INVENTION

The present invention is in the field of supplements. In particular, supplements containing alpha-GPC used for weight loss, and/or weight maintenance, and/or increase in lean body mass and/or maintenance of lean body mass in humans and animals.

### BACKGROUND OF THE DISCLOSURE

Obesity (defined as a body mass index (BMI) ≧30 kg/m2) affects approximately one-third of the U.S. population, and its prevalence has doubled over the past three decades. Prevalence of overweight (defined as a BMI ≧25 kg/m2) among adults in the U.S. has reached 68%. In 2008, an estimated 1.46 billion adults globally were overweight and 502 million adults were obese. Obesity predisposes humans to a range of serious medical conditions including hypertension, type 2 diabetes, dyslipidemia, and obstructive sleep apnea. Many of these diseases can be prevented or ameliorated with a reduction in body weight. Obesity has been identified as the second most common factor contributing to preventable death (second only to tobacco). In addition, the economic cost of obesity-associated diseases is nearly $100 billion per year in the U.S.

L-alpha glycerylphosphorylcholine (alpha-GPC) is a compound that is involved in phospholipid metabolism in animals, including humans. It is a metabolic precursor for membrane phospholipids. In addition, alpha-GPC crosses the blood brain barrier and is associated with increasing acetylcholine levels. Clinical studies with alpha-GPC indicate it may be useful for improving cognition in patients with dementia. Other studies indicate it may help accelerate recovery of patients who have had a stroke. Studies have also shown that alpha-GPC increases growth hormone secretion in humans.

Alpha-GPC is also known as Alfa-GPC, Alpha Glycerol Phosphoryl Choline, Alpha-Glyceryl Phosphoryl Choline, Alpha-Glyceryl Phosphatidylcholine, Alpha-glycerylphosphorylcholine, Choline alphoscerate, Glycerophosphorylcholine, Glycerophosphorylcholine, GPC, GroPCho, L-A-Glyceryl Phosphorylcholine, L-alpha-glycerylphosphorylcholine.

### SUMMARY OF THE INVENTION

The disclosure herein describes the use of alpha-GPC for weight loss, and/or weight maintenance and/or increasing or maintaining lean body mass, and formulations of alpha-GPC useful for weight loss, weight maintenance and/or increasing or maintaining lean body mass. These formulations include alpha-GPC alone as the active ingredient or combined with naturally derived compounds, synthetic compounds and plant extracts. The combination of alpha-GPC with these other compounds and extracts may result in complementary and synergistic pharmaceutical effects when compared to the effects of the individual components used alone with respect to weight loss and increased lean body mass.

An embodiment of the dietary supplements disclosed herein comprises alpha-GPC and one or more of D-aspartic acid, 7-keto DHEA, forskolin standardized to about 10-20%, EGCG, caffeine, conjugated linoleic acids (CLA), raspberry ketone, green tea extract, resveratrol, L-carnitine, coconut oil, hoodia, white bean extract, fucoxanthin, cissus quadrangularis, coleus forskohlii, and fiber. In another embodiment of this dietary supplement or formulation, the formulation comprises alpha-GPC, D-aspartic acid and 7-keto DHEA and forskolin, for example, without limitation, about 800 mg of alpha-GPC, about 3 g D-aspartic acid, about 200 mg of 7-Keto DHEA, and about 500 mg forskolin standardized to 10-20% per serving. Another embodiment of an alpha-GPC formulation comprises alpha-GPC, EGCG, 7-Keto DHEA and forskolin, for example, without limitation, about 800 mg of alpha-GPC, about 400 mg EGCG, about 200 mg of 7-Keto DHEA, and about 500 mg forskolin standardized to 10-20% per serving.

Also disclosed herein are methods for weight loss and/or weight maintenance and/or preserving or increasing lean body mass comprising the administration of alpha glycerylphosphorylcholine (alpha-GPC) as the sole active ingredient and/or in combination with ingredients selected from one or more of D-aspartic acid, 7-keto DHEA, forskolin standardized to about 10-20%, EGCG (epigallocatechin gallate), caffeine, CLA (conjugated linoleic acid), raspberry ketone, green tea extract, resveratrol, L-carnitine, coconut oil, hoodia, white bean extract, fucoxanthin, cissus quadrangularis, coleus forskohlii, and fiber to a human or animal in need of losing or maintaining weight and/or gaining or maintaining lean muscle mass.

An embodiment of the methods disclosed herein for promoting fat loss or maintaining weight while preserving or increasing lean body mass comprises administering alpha-GPC alone and/or in combination with one or more of EGCG, 7-Keto DHEA, and forskolin standardized to about 10-20% to a human or animal in need of losing or maintaining weight and/or gaining or maintaining lean muscle mass. In an embodiment of this method, the amount of EGCG is about 400 mg, the amount of 7-keto DHEA is about 200 mg, the amount of alpha-GPC is about 800 mg and the amount of forskolin is about 500 mg standardized to 10-20% per serving.

In yet another embodiment of the methods disclosed herein, the alpha-GPC administered is combined with one or more of D-aspartic acid, 7-Keto DHEA, and forskolin standardized to about 10-20%. In an embodiment of this method, the amount of alpha-GPC administered is about 800 mg, the amount of D-aspartic acid is about 3 g, the amount of 7-keto DHEA is about 200 mg and the amount of forskolin is about 500 mg standardized to aboutl0-20% per serving.

In embodiments of the methods disclosed herein, the alpha-GPC is taken (ingested) or administered in a dose of about 100 mg to about 5000 mg per day. In yet other embodiments, the dose of alpha-GPC is about 300 mg to about 1200 mg per day. In still other embodiments, the dose of alpha-GPC is about 800 mg to about 2400 mg per day. Included in these dose ranges are all possible doses in the range, e.g. 100 mg-5000 mg would include, without limitation, 1000-1200 mg, or 500-700 mg and the like.

### DETAILED DESCRIPTION OF THE EMBODIMENTS

Described herein are dietary supplements comprising glycerylphosphorylcholine (alpha-GPC) used alone or combined with other compounds and plant extracts. These alpha-GPC supplements are useful in methods for weight loss, weight maintenance and/or increasing or maintaining lean body mass. Thus, alpha-GPC helps reduce excess body fat while minimizing the loss of lean tissue. Lean body mass can be defined as body weight−(body weight×body fat %).

The supplements disclosed herein comprise alpha-GPC alone or in combination with one or more of D-aspartic acid, 7-keto DHEA, forskolin standardized to about 10-20%, EGCG, caffeine or caffeine containing products, e.g., without limitation guarana, CLA, raspberry ketone, green tea extract, resveratrol, L-carnitine, coconut oil, hoodia, white bean extract, fucoxanthin, cissus quadrangularis, coleus forskohlii, and fiber or other compounds involved with weight loss and/or increasing lean body mass.

The combination of alpha-GPC with other know compounds associated with fat loss and/or maintenance and/or increase in lean body mass is expected to be synergistic when compared with the weight loss and/or weight maintenance and/or increase in lean body mass with such compounds in the absence of alpha-GPC.

An embodiment of a formulation disclosed herein comprises about 800 mg of alpha-GPC, about 400 mg of EGCG, about 200 mg of 7-Keto DHEA and about 500 mg of forskolin standardized to 10-20% per serving.

Another embodiment of a formulation disclosed herein comprises about 800 mg alpha-GPC, about 3 grams D-aspartic acid, about 200 mg of 7-keto DHEA and about 500 mg of forskolin standardized to 10-20% per serving.

The formulations can be taken one or more times daily. In some embodiments the amount of alpha-GPC taken by a subject for the methods disclosed herein is about 10 mg to about 10,000 mg per day. In other embodiments of the methods disclosed herein, the amount of alpha-GPC taken by a subject to achieve weight loss and/or lean body mass is about 100 mg to about 5000 mg per day. In yet other embodiments, the amount of alpha-GPC taken by a subject in need of losing weight and/or increasing and or maintaining lean body mass is about 400 mg to about 4000 mg per day or about 400 mg to about 3000 mg per day, or about 400 mg to about 2400 mg per day, or about 400 mg to about 2000 mg per day, or about 200 mg to about 4000 mg per day or about 200 mg to about 2000 mg per day or about 200 mg to about 1000 mg per day or about 300 mg to about 1200 mg per day or about 800 mg-3200 mg per day. The formulations disclosed herein may be in powder form, pill form or capsule form. If in powder form, the formulation can be added to a drinkable liquid such as water, juice, smoothie and the like or the powder can be added to food, e.g., without limitation oatmeal or yogurt.

The preparation of alpha-GPC is known in the art. For example, without limitation, alpha-GPC can prepared as described in US Publication No. 2012/0244583 and methods cited therein.

The methods and formulations described herein can be used for weight loss, or weight maintenance, or increasing lean body mass, or maintaining lean body mass or combinations of the foregoing in humans and animals, for example, the methods and formulations can be used for weight loss and increasing lean body mass, or weight maintenance and increasing lean body mass and so forth.

### EXAMPLES

The effect of alpha-GPC formulations on body composition and metabolic parameters are studied in a double-blind randomized placebo-controlled study. The supplement formulations and/or alpha-GPC used alone are tested using two groups, Group I and Group II consisting of 5-100 or more subjects. Group I are administered a placebo or supplement containing various components of the formulations disclosed herein without alpha-GPC for 12 days and thereafter for 13 days receive the supplement containing alpha-GPC. Data are determined for average weight loss per person, average weight lost per person per week, percent body fat, basal metabolic rate, average BMI reduction per person, average waist size reduction, total cholesterol, triglycerides, and glucose.

A number of embodiments of the invention have been described. Nevertheless, it will be understood that various modifications may be made without departing from the spirit and scope of the invention. Accordingly, other embodiments are within the scope of the following claims.

What is claimed is:

1. A method of promoting weight loss and/or maintaining weight and/or maintaining and/or increasing lean body mass comprising the administration of alpha glycerylphosphorylcholine (alpha-GPC) alone or in combination with ingredients selected from one or more of D-aspartic acid, 7-keto DHEA, forskolin standardized to about 10-20%, EGCG, caffeine, CLA, raspberry ketone, green tea extract, resveratrol, L-carnitine, coconut oil, hoodia, white bean extract, fucoxanthin, cissus quadrangularis, coleus forskohlii, and fiber to a human or animal in need of maintaining and/or losing weight and/or increasing or maintaining lean muscle mass.

2. The method according to claim 1, wherein the alpha-GPC is combined with EGCG, 7-Keto DHEA, and forskolin standardized to about 10-20%.

3. The method of claim 2, wherein the amount of EGCG is about 400 mg, the amount of 7-keto DHEA is about 200 mg, the amount of alpha-GPC is about 800 mg and the amount of forskolin is about 500 mg of 10-20% standardized to about 10-20% per serving.

4. The method according to claim 1, wherein the alpha-GPC is combined with D-aspartic acid, 7-Keto DHEA, and forskolin standardized to 10-20%.

5. The method according to claim 4, wherein the amount of alpha-GPC is about 800 mg, the amount of D-aspartic acid is about 3 g, the amount of 7-keto DHEA is about 200 mg and the amount of forskolin is about 500 mg standardized to 10-20% per serving.

6. A supplement for maintaining weight and/or promoting weight loss and/or maintaining lean body mass and/or increasing lean body mass comprising alpha-GPC and one or more compounds involved in maintaining and/or increasing lean body mass and/or maintaining and/or promoting weight loss.

7. The supplement of claim 6 comprising about 800 mg of alpha-GPC, about 400 mg EGCG, about 200 mg of 7-Keto DHEA, and about 500 mg forskolin standardized to 10-20% per serving.

8. The supplement of claim 6 comprising about 800 mg of alpha-GPC, about 3 g D-aspartic acid, about 200 mg of 7-Keto DHEA, and about 500 mg forskolin standardized to 10-20% per serving.

9. The method according to claim 1, wherein the alpha-GPC is taken in a dose of about 100 mg to about 5000 mg per day.

10. The method according to claim 9, wherein the dose of alpha-GPC is about 300 mg to about 1200 mg per day.

11. The method according to claim 9, wherein the dose of alpha-GPC is about 800 mg to about 2400 mg per day.

12. The supplement of claim 6 comprising alpha-GPC and one or more of D-aspartic acid, 7-keto DHEA, forskolin standardized to about 10-20%, EGCG, caffeine, CLA, raspberry ketone, green tea extract, resveratrol, L-carnitine, coconut oil, hoodia, white bean extract, fucoxanthin, cissus quadrangularis, coleus forskohlii, and fiber.

13. A method for increasing lean body mass comprising the administration of alpha glycerylphosphorylcholine (alpha-GPC) alone or in combination with ingredients selected from one or more of D-aspartic acid, 7-keto DHEA, forskolin standardized to about 10-20%., EGCG, caffeine, CLA, raspberry ketone, green tea extract, resveratrol, L-carnitine, coconut oil, hoodia, white bean extract, fucoxanthin, cissus quadrangularis, coleus forskohlii, and fiber to a human or animal in need of increasing lean muscle mass.
